(19) United States
(12) Patent Application Publication
(10) Pub. No.: US 2021/0085558 A1
(43) Pub. Date: Mar. 25, 2021

(54) ARTIFICIAL INTELLIGENCE MASSAGE APPARATUS AND METHOD FOR CONTROLLING MASSAGE OPERATION IN CONSIDERATION OF FACIAL EXPRESSION OR UTTERANCE OF USER

(71) Applicant: LG ELECTRONICS INC., Seoul (KR)

(72) Inventors: Wonho SHIN, Seoul (KR); Jichan MAENG, Seoul (KR)

(73) Assignee: LG ELECTRONICS INC., Seoul (KR)

(21) Appl. No.: 16/664,533

(22) Filed: Oct. 25, 2019

(30) Foreign Application Priority Data
Sep. 24, 2019 (KR) 10-2019-0117704

(51) Int. Cl. A61H 23/02; G06N 3/08; G10L 15/22; G06K 9/00; G10L 25/63

(52) U.S. Cl. CPC A61H 23/02; G06N 3/08; G10L 15/22; G06K 9/00315; A61H 2201/0149; G06K 9/00241; G10L 25/63; G10L 2015/223; A61H 2201/501; G06K 9/00275

(57) ABSTRACT

An artificial intelligence massage apparatus for controlling a massage operation according to an embodiment of the present disclosure includes a microphone, a camera, a driver comprising at least one motor, and a processor configured to obtain image data including a face of a user via the camera, determine whether the user is uttering using the obtained image data, obtain speech data via the microphone, if it is determined that the user is uttering, generate intention information corresponding to the obtained speech data, and control a massage operation based on the generated intention information by controlling the driver.

### CROSS-REFERENCE TO RELATED APPLICATIONS

This application claims priority to Korean Patent Application No. 10-2019-0117704 filed in the Republic of Korea on Sep. 24, 2019, the entire contents of which are hereby incorporated by reference in its entirety.

### BACKGROUND

The present disclosure relates to an artificial intelligence massage apparatus and a method for controlling a massage operation in consideration of a facial expression or utterance of a user. In particular, the present disclosure relates to an artificial intelligence massage apparatus and a method for controlling a massage operation by recognizing the motion of the user, facial expression or utterance content while providing a massage operation.

Recently, there is a growing demand for massage equipment due to the high demand to loosen the clumped muscles or to relieve fatigue and stress via the massage. Massage is a type of medical adjuvant therapy that involves sweeping, kneading, pressing, pulling, knocking, or moving a body with hands or a special device to aid blood circulation and relieve fatigue. An apparatus for performing massage by a mechanical device is called a massage apparatus, and a typical example of the massage apparatus may be a massage chair in which a user can comfortably sit and receive a massage.

Meanwhile, if the strength of the massage is too strong, the massage may cause pain to the user. In this case, the user needs to lower the intensity of the massage, but there is an inconvenience to the user that a button operation using an input device mounted on the massage chair or a remote controller is performed in order to reduce the intensity of the massage.

### SUMMARY

The present disclosure is to provide an artificial intelligence massage apparatus and a method for providing a massage operation suitable for the user in consideration of the facial expression or utterance of the user.

According to an embodiment of the present disclosure, there is provided an artificial intelligence massage apparatus and a method thereof which obtains image data including a face of a user, determines whether a user is uttering using the obtained image data, obtains speech data via a microphone if it is determined that the user is uttering, generates intention information corresponding to the obtained speech data, and controls a massage operation based on the intention information.

In addition, according to an embodiment of the present disclosure, there is provided an artificial intelligence massage apparatus and a method thereof which, if the user is not uttering, determines an object massage operation using the obtained image data and massage operation information and controls the massage operation based on the determined object massage operation.

According to an embodiment of the present disclosure, there is provided an artificial intelligence massage apparatus and a method thereof which generates facial expression information from obtained image data using a facial expression information generation model, and determines an object massage operation from facial expression information and massage operation information generated using the first massage operation determination model.

In addition, according to an embodiment of the present disclosure, there is provided an artificial intelligence massage apparatus and a method thereof which determines an object massage operation from the image data obtained using a second massage operation determination model.

In addition, according to an embodiment of the present disclosure, there is provided an artificial intelligence massage apparatus and a method thereof which, if the massage operation is controlled based on the intention information generated from the speech data, generates training data including control information of the massage operation corresponding to the image data or the facial expression information, the massage operation information, and the generated intention information.

### BRIEF DESCRIPTION OF THE DRAWINGS

FIG. 1 is a block diagram illustrating an AI apparatus according to an embodiment of the present disclosure.

FIG. 2 is a block diagram illustrating an AI server according to an embodiment of the present disclosure.

FIG. 3 is a view illustrating an AI system according to an embodiment of the present disclosure.

FIG. 4 is a block diagram illustrating an AI apparatus according to an embodiment of the present disclosure.

FIG. 5 is a perspective view illustrating an artificial intelligence apparatus according to an embodiment of the present disclosure.

FIG. 6 is a block diagram illustrating an AI system according to an embodiment of the present disclosure.

FIG. 7 is a flowchart illustrating a method for controlling a massage operation in consideration of a facial expression or utterance of a user according to an embodiment of the present disclosure.

FIG. 8 is a view illustrating a method for determining an object massage operation by using a first massage operation determination model according to an embodiment of the present disclosure.

FIG. 9 is a flowchart illustrating a method for controlling a massage operation in consideration of a facial expression or utterance of a user according to an exemplary embodiment of the present disclosure.

FIG. 10 is a view illustrating a method of determining an object massage operation using a second massage gesture determination model according to an exemplary embodiment of the present disclosure.

FIGS. 11 and 12 are views illustrating examples of controlling a massage operation in consideration of the facial expression or utterance of the user according to an embodiment of the present disclosure.

FIGS. 13 and 14 are views illustrating examples of controlling a massage operation in consideration of the facial expression or utterance of the user according to an embodiment of the present disclosure.

FIGS. 15 and 16 are views illustrating examples of controlling a massage operation in consideration of the facial expression or utterance of the user according to an embodiment of the present disclosure.

FIG. 17 is a view illustrating an example of controlling a massage operation in consideration of a change in the facial expression of a user according to an embodiment of the present disclosure.

FIG. 18 is a view illustrating an example of controlling a massage operation in consideration of a change in the facial expression of a user according to an embodiment of the present disclosure.

FIG. 19 is a view illustrating an example of controlling a massage operation according to an embodiment of the present disclosure.

### DETAILED DESCRIPTION OF THE EMBODIMENTS

Hereinafter, embodiments of the present disclosure are described in more detail with reference to accompanying drawings and regardless of the drawings symbols, same or similar components are assigned with the same reference numerals and thus overlapping descriptions for those are omitted. The suffixes “module” and “unit” for components used in the description below are assigned or mixed in consideration of easiness in writing the specification and do not have distinctive meanings or roles by themselves. In the following description, detailed descriptions of well-known functions or constructions will be omitted since they would obscure the disclosure in unnecessary detail. Additionally, the accompanying drawings are used to help easily understanding embodiments disclosed herein but the technical idea of the present disclosure is not limited thereto. It should be understood that all of variations, equivalents or substitutes contained in the concept and technical scope of the present disclosure are also included.

It will be understood that the terms “first” and “second” are used herein to describe various components but these components should not be limited by these terms. These terms are used only to distinguish one component from other components.

In this disclosure below, when one part (or element, device, etc.) is referred to as being ‘connected’ to another part (or element, device, etc.), it should be understood that the former can be ‘directly connected’ to the latter, or ‘electrically connected’ to the latter via an intervening part (or element, device, etc.). It will be further understood that when one component is referred to as being ‘directly connected’ or ‘directly linked’ to another component, it means that no intervening component is present.

<Artificial Intelligence (AI)>

Artificial intelligence refers to the field of studying artificial intelligence or methodology for making artificial intelligence, and machine learning refers to the field of defining various issues dealt with in the field of artificial intelligence and studying methodology for solving the various issues. Machine learning is defined as an algorithm that enhances the performance of a certain task through a steady experience with the certain task.

An artificial neural network (ANN) is a model used in machine learning and may mean a whole model of problem-solving ability which is composed of artificial neurons (nodes) that form a network by synaptic connections. The artificial neural network can be defined by a connection pattern between neurons in different layers, a learning process for updating model parameters, and an activation function for generating an output value.

The artificial neural network may include an input layer, an output layer, and optionally one or more hidden layers. Each layer includes one or more neurons, and the artificial neural network may include a synapse that links neurons to neurons. In the artificial neural network, each neuron may output the function value of the activation function for input signals, weights, and deflections input through the synapse.

Model parameters refer to parameters determined through learning and include a weight value of synaptic connection and deflection of neurons. A hyperparameter means a parameter to be set in the machine learning algorithm before learning, and includes a learning rate, a repetition number, a mini batch size, and an initialization function.

The purpose of the learning of the artificial neural network may be to determine the model parameters that minimize a loss function. The loss function may be used as an index to determine optimal model parameters in the learning process of the artificial neural network.

Machine learning may be classified into supervised learning, unsupervised learning, and reinforcement learning according to a learning method.

The supervised learning may refer to a method of learning an artificial neural network in a state in which a label for training data is given, and the label may mean the correct answer (or result value) that the artificial neural network must infer when the training data is input to the artificial neural network. The unsupervised learning may refer to a method of learning an artificial neural network in a state in which a label for training data is not given. The reinforcement learning may refer to a learning method in which an agent defined in a certain environment learns to select a behavior or a behavior sequence that maximizes cumulative compensation in each state.

Machine learning, which is implemented as a deep neural network (DNN) including a plurality of hidden layers among artificial neural networks, is also referred to as deep learning, and the deep learning is part of machine learning. In the following, machine learning is used to mean deep learning

<Robot>

A robot may refer to a machine that automatically processes or operates a given task by its own ability. In particular, a robot having a function of recognizing an environment and performing a self-determination operation may be referred to as an intelligent robot.

Robots may be classified into industrial robots, medical robots, home robots, military robots, and the like according to the use purpose or field.

The robot includes a driving unit may include an actuator or a motor and may perform various physical operations such as moving a robot joint. In addition, a movable robot may include a wheel, a brake, a propeller, and the like in a driving unit, and may travel on the ground through the driving unit or fly in the air.

<Self-Driving>

Self-driving refers to a technique of driving for oneself, and a self-driving vehicle refers to a vehicle that travels without an operation of a user or with a minimum operation of a user.

For example, the self-driving may include technology for maintaining a lane while driving, a technology for automatically adjusting a speed, such as adaptive cruise control, a technique for automatically traveling along a predetermined route, and technology for automatically setting and traveling a route when a destination is set.

The vehicle may include a vehicle having only an internal combustion engine, a hybrid vehicle having an internal combustion engine and an electric motor together, and an electric vehicle having only an electric motor, and may include not only an automobile but also a train, a motorcycle, and the like.

Here, the self-driving vehicle may be regarded as a robot having a self-driving function.

<EXtended Reality (XR)>

Extended reality is collectively referred to as virtual reality (VR), augmented reality (AR), and mixed reality (MR). The VR technology provides a real-world object and background only as a CG image, the AR technology provides a virtual CG image on a real object image, and the MR technology is a computer graphic technology that mixes and combines virtual objects into the real world.

The MR technology is similar to the AR technology in that the real object and the virtual object are shown together. However, in the AR technology, the virtual object is used in the form that complements the real object, whereas in the MR technology, the virtual object and the real object are used in an equal manner.

The XR technology may be applied to a head-mount display (HMD), a head-up display (HUD), a mobile phone, a tablet PC, a laptop, a desktop, a TV, a digital signage, and the like. A device to which the XR technology is applied may be referred to as an XR device.

Hereinafter, the AI massage apparatus may be referred to as an artificial intelligence apparatus, and the two terms may be used interchangeably.

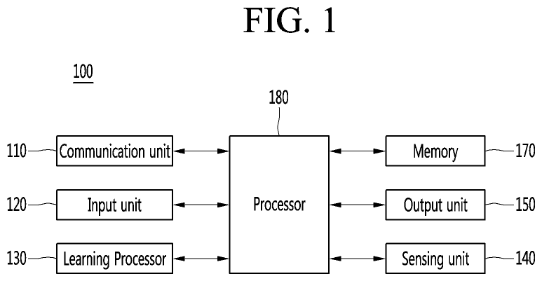

FIG. 1 is a block diagram illustrating an AI apparatus 100 according to an embodiment of the present disclosure.

Hereinafter, the AI apparatus 100 may be referred to as a terminal.

The AI apparatus (or an AI device) 100 may be implemented by a stationary device or a mobile device, such as a TV, a projector, a mobile phone, a smartphone, a desktop computer, a notebook, a digital broadcasting terminal, a personal digital assistant (PDA), a portable multimedia player (PMP), a navigation device, a tablet PC, a wearable device, a set-top box (STB), a DMB receiver, a radio, a washing machine, a refrigerator, a desktop computer, a digital signage, a robot, a vehicle, and the like.

Referring to FIG. 1, the AI apparatus 100 may include a communication unit 110, an input unit 120, a learning processor 130, a sensing unit 140, an output unit 150, a memory 170, and a processor 180.

The communication unit 110 may transmit and receive data to and from external devices such as other 100a to 100e and the AI server 200 by using wire/wireless communication technology. For example, the communication unit 110 may transmit and receive sensor information, a user input, a learning model, and a control signal to and from external devices.

The communication technology used by the communication unit 110 includes GSM (Global System for Mobile communication), CDMA (Code Division Multi Access), LTE (Long Term Evolution), 5G, WLAN (Wireless LAN), Wi-Fi (Wireless-Fidelity), Bluetooth™, RFID (Radio Frequency Identification), Infrared Data Association (IrDA), ZigBee, NFC (Near Field Communication), and the like.

The input unit 120 may acquire various kinds of data.

Here, the input unit 120 may include a camera for inputting a video signal, a microphone for receiving an audio signal, and a user input unit for receiving information from a user. The camera or the microphone may be treated as a sensor, and the signal obtained from the camera or the microphone may be referred to as sensing data or sensor information.

The input unit 120 may acquire a training data for model learning and an input data to be used when an output is obtained by using learning model. The input unit 120 may acquire raw input data. Here, the processor 180 or the learning processor 130 may extract an input feature by preprocessing the input data.

The learning processor 130 may learn a model composed of an artificial neural network by using training data. The learned artificial neural network may be referred to as a learning model. The learning model may be used to an infer result value for new input data rather than training data, and the inferred value may be used as a basis for determination to perform a certain operation.

Here, the learning processor 130 may perform AI processing together with the learning processor 240 of the AI server 200.

Here, the learning processor 130 may include a memory integrated or implemented in the AI apparatus 100. Alternatively, the learning processor 130 may be implemented by using the memory 170, an external memory directly connected to the AI apparatus 100, or a memory held in an external device.

The sensing unit 140 may acquire at least one of internal information about the AI apparatus 100, ambient environment information about the AI apparatus 100, and user information by using various sensors.

Examples of the sensors included in the sensing unit 140 may include a proximity sensor, an illuminance sensor, an acceleration sensor, a magnetic sensor, a gyro sensor, an inertial sensor, an RGB sensor, an IR sensor, a fingerprint recognition sensor, an ultrasonic sensor, an optical sensor, a microphone, a lidar, and a radar.

The output unit 150 may generate an output related to a visual sense, an auditory sense, or a haptic sense.

Here, the output unit 150 may include a display unit for outputting time information, a speaker for outputting auditory information, and a haptic module for outputting haptic information.

The memory 170 may store data that supports various functions of the AI apparatus 100. For example, the memory 170 may store input data obtained by the input unit 120, training data, a learning model, a learning history, and the like.

The processor 180 may determine at least one executable operation of the AI apparatus 100 based on information determined or generated by using a data analysis algorithm or a machine learning algorithm. The processor 180 may control the components of the AI apparatus 100 to execute the determined operation.

To this end, the processor 180 may request, search, receive, or utilize data of the learning processor 130 or the memory 170. The processor 180 may control the components of the AI apparatus 100 to execute the predicted operation or the operation determined to be desirable among the at least one executable operation.

When the connection of an external device is required to perform the determined operation, the processor 180 may generate a control signal for controlling the external device and may transmit the generated control signal to the external device.

The processor 180 may acquire intention information for the user input and may determine the user's requirements based on the obtained intention information.

The processor 180 may acquire the intention information corresponding to the user input by using at least one of a speech to text (STT) engine for converting speech input into a text string or a natural language processing (NLP) engine for acquiring intention information of a natural language.

At least one of the STT engine or the NLP engine may be configured as an artificial neural network, at least part of which is learned according to the machine learning algorithm. At least one of the STT engine or the NLP engine may be learned by the learning processor 130, may be learned by the learning processor 240 of the AI server 200, or may be learned by their distributed processing.

The processor 180 may collect history information including the operation contents of the AI apparatus 100 or the user's feedback on the operation and may store the collected history information in the memory 170 or the learning processor 130 or transmit the collected history information to the external device such as the AI server 200. The collected history information may be used to update the learning model.

The processor 180 may control at least part of the components of AI apparatus 100 to drive an application program stored in memory 170. Furthermore, the processor 180 may operate two or more of the components included in the AI apparatus 100 in combination to drive the application program.

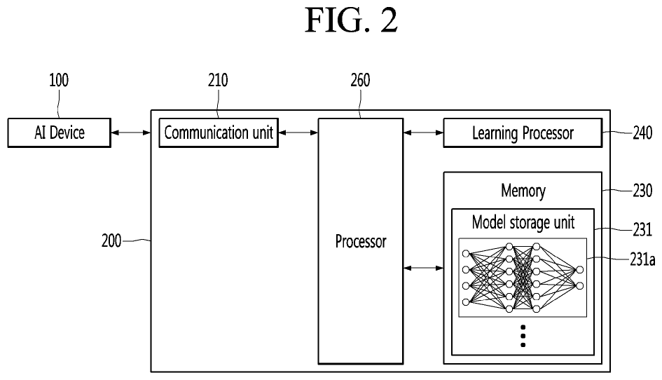

FIG. 2 is a block diagram illustrating an AI server 200 according to an embodiment of the present disclosure.

Referring to FIG. 2, the AI server 200 may refer to a device that learns an artificial neural network by using a machine learning algorithm or uses a learned artificial neural network. The AI server 200 may include a plurality of servers to perform distributed processing, or may be defined as a 5G network. Here, the AI server 200 may be included as a partial configuration of the AI apparatus 100, and may perform at least part of the AI processing together.

The AI server 200 may include a communication unit 210, a memory 230, a learning processor 240, a processor 260, and the like.

The communication unit 210 can transmit and receive data to and from an external device such as the AI apparatus 100.

The memory 230 may include a model storage unit 231. The model storage unit 231 may store a learning or learned model (or an artificial neural network 231a) through the learning processor 240.

The learning processor 240 may learn the artificial neural network 231a by using the training data. The learning model may be used in a state of being mounted on the AI server 200 of the artificial neural network, or may be used in a state of being mounted on an external device such as the AI apparatus 100.

The learning model may be implemented in hardware, software, or a combination of hardware and software. If all or part of the learning models are implemented in software, one or more instructions that constitute the learning model may be stored in memory 230.

The processor 260 may infer the result value for new input data by using the learning model and may generate a response or a control command based on the inferred result value.

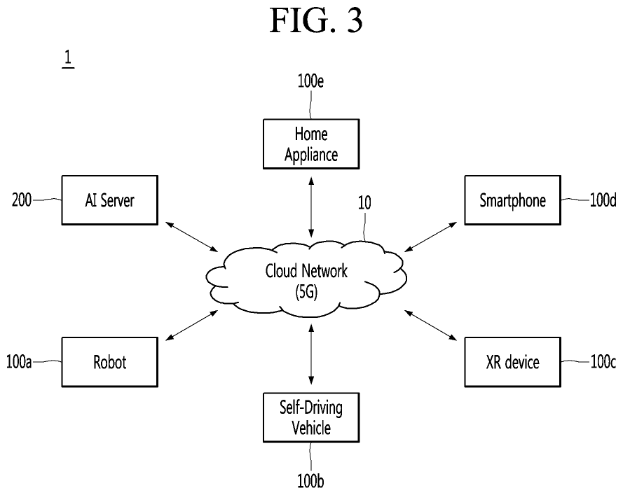

FIG. 3 is a view illustrating an AI system 1 according to an embodiment of the present disclosure.

Referring to FIG. 3, in the AI system 1, at least one of an AI server 200, a robot 100a, a self-driving vehicle 100b, an XR device 100c, a smartphone 100d, or a home appliance 100e is connected to a cloud network 10. The robot 100a, the self-driving vehicle 100b, the XR device 100c, the smartphone 100d, or the home appliance 100e, to which the AI technology is applied, may be referred to as AI apparatuses 100a to 100e.

The cloud network 10 may refer to a network that forms part of a cloud computing infrastructure or exists in a cloud computing infrastructure. The cloud network 10 may be configured by using a 3G network, a 4G or LTE network, or a 5G network.

In other words, the devices 100a to 100e and 200 configuring the AI system 1 may be connected to each other through the cloud network 10. In particular, each of the devices 100a to 100e and 200 may communicate with each other through a base station, but may directly communicate with each other without using a base station.

The AI server 200 may include a server that performs AI processing and a server that performs operations on big data.

The AI server 200 may be connected to at least one of the AI apparatuses constituting the AI system 1, that is, the robot 100a, the self-driving vehicle 100b, the XR device 100c, the smartphone 100d, or the home appliance 100e through the cloud network 10, and may assist at least part of AI processing of the connected AI apparatuses 100a to 100e.

Here, the AI server 200 may learn the artificial neural network according to the machine learning algorithm instead of the AI apparatuses 100a to 100e, and may directly store the learning model or transmit the learning model to the AI apparatuses 100a to 100e.

Here, the AI server 200 may receive input data from the AI apparatuses 100a to 100e, may infer the result value for the received input data by using the learning model, may generate a response or a control command based on the inferred result value, and may transmit the response or the control command to the AI apparatuses 100a to 100e.

Alternatively, the AI apparatuses 100a to 100e may infer the result value for the input data by directly using the learning model, and may generate the response or the control command based on the inference result.

Hereinafter, various embodiments of the AI apparatuses 100a to 100e to which the above-described technology is applied will be described. The AI apparatuses 100a to 100e illustrated in FIG. 3 may be regarded as a specific embodiment of the AI apparatus 100 illustrated in FIG. 1.

<AI+Robot>

The robot 100a, to which the AI technology is applied, may be implemented as a guide robot, a carrying robot, a cleaning robot, a wearable robot, an entertainment robot, a pet robot, an unmanned flying robot, or the like.

The robot 100a may include a robot control module for controlling the operation, and the robot control module may refer to a software module or a chip implementing the software module by hardware.

The robot 100a may acquire state information about the robot 100a by using sensor information obtained from various kinds of sensors, may detect (recognize) surrounding environment and objects, may generate map data, may determine the route and the travel plan, may determine the response to user interaction, or may determine the operation.

The robot 100a may use the sensor information obtained from at least one sensor among the lidar, the radar, and the camera to determine the travel route and the travel plan.

The robot 100a may perform the above-described operations by using the learning model composed of at least one artificial neural network. For example, the robot 100a may recognize the surrounding environment and the objects by using the learning model, and may determine the operation by using the recognized surrounding information or object information. The learning model may be learned directly from the robot 100a or may be learned from an external device such as the AI server 200.

Here, the robot 100a may perform the operation by generating the result by directly using the learning model, but the sensor information may be transmitted to the external device such as the AI server 200 and the generated result may be received to perform the operation.

The robot 100a may use at least one of the map data, the object information detected from the sensor information, or the object information obtained from the external device to determine the travel route and the travel plan, and may control the driving unit such that the robot 100a travels along the determined travel route and travel plan.

The map data may include object identification information about various objects arranged in the space in which the robot 100a moves. For example, the map data may include object identification information about fixed objects such as walls and doors and movable objects such as pollen and desks. The object identification information may include a name, a type, a distance, and a position.

In addition, the robot 100a may perform the operation or travel by controlling the driving unit based on the control/interaction of the user. Here, the robot 100a may acquire the intention information of the interaction due to the user's operation or speech utterance, and may determine the response based on the obtained intention information, and may perform the operation.

<AI+Self-Driving>

The self-driving vehicle 100b, to which the AI technology is applied, may be implemented as a mobile robot, a vehicle, an unmanned flying vehicle, or the like.

The self-driving vehicle 100b may include a self-driving control module for controlling a self-driving function, and the self-driving control module may refer to a software module or a chip implementing the software module by hardware. The self-driving control module may be included in the self-driving vehicle 100b as a component thereof, but may be implemented with separate hardware and connected to the outside of the self-driving vehicle 100b.

The self-driving vehicle 100b may acquire state information about the self-driving vehicle 100b by using sensor information obtained from various kinds of sensors, may detect (recognize) surrounding environment and objects, may generate map data, may determine the route and the travel plan, or may determine the operation.

Like the robot 100a, the self-driving vehicle 100b may use the sensor information obtained from at least one sensor among the lidar, the radar, and the camera to determine the travel route and the travel plan.

In particular, the self-driving vehicle 100b may recognize the environment or objects for an area covered by a field of view or an area over a certain distance by receiving the sensor information from external devices, or may receive directly recognized information from the external devices.

The self-driving vehicle 100b may perform the above-described operations by using the learning model composed of at least one artificial neural network. For example, the self-driving vehicle 100b may recognize the surrounding environment and the objects by using the learning model, and may determine the traveling route by using the recognized surrounding information or object information. The learning model may be learned directly from the self-driving vehicle 100a or may be learned from an external device such as the AI server 200.

Here, the self-driving vehicle 100b may perform the operation by generating the result by directly using the learning model, but the sensor information may be transmitted to the external device such as the AI server 200 and the generated result may be received to perform the operation.

The self-driving vehicle 100b may use at least one of the map data, the object information detected from the sensor information, or the object information obtained from the external device to determine the travel route and the travel plan, and may control the driving unit such that the self-driving vehicle 100b travels along the determined travel route and travel plan.

The map data may include object identification information about various objects arranged in the space (for example, road) in which the self-driving vehicle 100b travels. For example, the map data may include object identification information about fixed objects such as street lamps, rocks, and buildings and movable objects such as vehicles and pedestrians. The object identification information may include a name, a type, a distance, and a position.

In addition, the self-driving vehicle 100b may perform the operation or travel by controlling the driving unit based on the control/interaction of the user. Here, the self-driving vehicle 100b may acquire the intention information of the interaction due to the user's operation or speech utterance, and may determine the response based on the obtained intention information, and may perform the operation.

<AI+XR>

The XR device 100c, to which the AI technology is applied, may be implemented by a head-mount display (HMD), a head-up display (HUD) provided in the vehicle, a television, a mobile phone, a smartphone, a computer, a wearable device, a home appliance, a digital signage, a vehicle, a fixed robot, a mobile robot, or the like.

The XR device 100c may analyzes three-dimensional point cloud data or image data obtained from various sensors or the external devices, generate position data and attribute data for the three-dimensional points, acquire information about the surrounding space or the real object, and render to output the XR object to be output. For example, the XR device 100c may output an XR object including the additional information about the recognized object in correspondence to the recognized object.

The XR device 100c may perform the above-described operations by using the learning model composed of at least one artificial neural network. For example, the XR device 100c may recognize the real object from the three-dimensional point cloud data or the image data by using the learning model, and may provide information corresponding to the recognized real object. The learning model may be directly learned from the XR device 100c, or may be learned from the external device such as the AI server 200.

Here, the XR device 100c may perform the operation by generating the result by directly using the learning model, but the sensor information may be transmitted to the external device such as the AI server 200 and the generated result may be received to perform the operation.

<AI+Robot+Self-Driving>

The robot 100a, to which the AI technology and the self-driving technology are applied, may be implemented as a guide robot, a carrying robot, a cleaning robot, a wearable robot, an entertainment robot, a pet robot, an unmanned flying robot, or the like.

The robot 100a, to which the AI technology and the self-driving technology are applied, may refer to the robot itself having the self-driving function or the robot 100a interacting with the self-driving vehicle 100b.

The robot 100a having the self-driving function may collectively refer to a device that moves for itself along the given route without the user's control or moves for itself by determining the route by itself

The robot 100a and the self-driving vehicle 100b having the self-driving function may use a common sensing method to determine at least one of the travel route or the travel plan. For example, the robot 100a and the self-driving vehicle 100b having the self-driving function may determine at least one of the travel route or the travel plan by using the information sensed through the lidar, the radar, and the camera.

The robot 100a that interacts with the self-driving vehicle 100b exists separately from the self-driving vehicle 100b and may perform operations interworking with the self-driving function of the self-driving vehicle 100b or interworking with the user who rides on the self-driving vehicle 100b.

Here, the robot 100a interacting with the self-driving vehicle 100b may control or assist the self-driving function of the self-driving vehicle 100b by acquiring sensor information on behalf of the self-driving vehicle 100b and providing the sensor information to the self-driving vehicle 100b, or by acquiring sensor information, generating environment information or object information, and providing the information to the self-driving vehicle 100b.

Alternatively, the robot 100a interacting with the self-driving vehicle 100b may monitor the user boarding the self-driving vehicle 100b, or may control the function of the self-driving vehicle 100b through the interaction with the user. For example, when it is determined that the driver is in a drowsy state, the robot 100a may activate the self-driving function of the self-driving vehicle 100b or assist the control of the driving unit of the self-driving vehicle 100b. The function of the self-driving vehicle 100b controlled by the robot 100a may include not only the self-driving function but also the function provided by the navigation system or the audio system provided in the self-driving vehicle 100b.

Alternatively, the robot 100a that interacts with the self-driving vehicle 100b may provide information or assist the function to the self-driving vehicle 100b outside the self-driving vehicle 100b. For example, the robot 100a may provide traffic information including signal information and the like, such as a smart signal, to the self-driving vehicle 100b, and automatically connect an electric charger to a charging port by interacting with the self-driving vehicle 100b like an automatic electric charger of an electric vehicle.

<AI+Robot+XR>

The robot 100a, to which the AI technology and the XR technology are applied, may be implemented as a guide robot, a carrying robot, a cleaning robot, a wearable robot, an entertainment robot, a pet robot, an unmanned flying robot, a drone, or the like.

The robot 100a, to which the XR technology is applied, may refer to a robot that is subjected to control/interaction in an XR image. In this case, the robot 100a may be separated from the XR device 100c and interwork with each other.

When the robot 100a, which is subjected to control/interaction in the XR image, may acquire the sensor information from the sensors including the camera, the robot 100a or the XR device 100c may generate the XR image based on the sensor information, and the XR device 100c may output the generated XR image. The robot 100a may operate based on the control signal input through the XR device 100c or the user's interaction.

For example, the user can confirm the XR image corresponding to the time point of the robot 100a interworking remotely through the external device such as the XR device 100c, adjust the self-driving travel path of the robot 100a through interaction, control the operation or driving, or confirm the information about the surrounding object.

<AI+Self-Driving+XR>

The self-driving vehicle 100b, to which the AI technology and the XR technology are applied, may be implemented as a mobile robot, a vehicle, an unmanned flying vehicle, or the like.

The self-driving driving vehicle 100b, to which the XR technology is applied, may refer to a self-driving vehicle having a means for providing an XR image or a self-driving vehicle that is subjected to control/interaction in an XR image. Particularly, the self-driving vehicle 100b that is subjected to control/interaction in the XR image may be distinguished from the XR device 100c and interwork with each other.

The self-driving vehicle 100b having the means for providing the XR image may acquire the sensor information from the sensors including the camera and output the generated XR image based on the obtained sensor information. For example, the self-driving vehicle 100b may include an HUD to output an XR image, thereby providing a passenger with a real object or an XR object corresponding to an object in the screen.

Here, when the XR object is output to the HUD, at least part of the XR object may be outputted to overlap the actual object to which the passenger's gaze is directed. Meanwhile, when the XR object is output to the display provided in the self-driving vehicle 100b, at least part of the XR object may be output to overlap the object in the screen. For example, the self-driving vehicle 100b may output XR objects corresponding to objects such as a lane, another vehicle, a traffic light, a traffic sign, a two-wheeled vehicle, a pedestrian, a building, and the like.

When the self-driving vehicle 100b, which is subjected to control/interaction in the XR image, may acquire the sensor information from the sensors including the camera, the self-driving vehicle 100b or the XR device 100c may generate the XR image based on the sensor information, and the XR device 100c may output the generated XR image. The self-driving vehicle 100b may operate based on the control signal input through the external device such as the XR device 100c or the user's interaction.

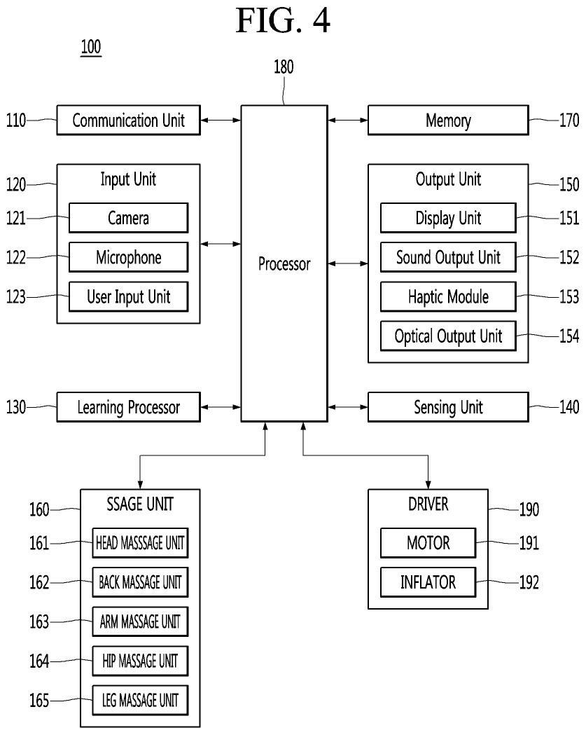

FIG. 4 is a block diagram illustrating an AI apparatus 100 according to an embodiment of the present disclosure.

The redundant repeat of FIG. 1 will be omitted below.

The communication unit 110 may also be referred to as a communicator.

Referring to FIG. 4, the artificial intelligence apparatus 100 may further include a driver 160.

The input unit 120 may include a camera 121 for image signal input, a microphone 122 for receiving audio signal input, and a user input unit 123 for receiving information from a user.

Speech data or image data collected by the input unit 120 are analyzed and processed as the user's control command.

Then, the input unit 120 is used for inputting image information (or signal), audio information (or signal), data, or information inputted from a user and the AI apparatus 100 may include at least one camera 121 in order for inputting image information.

The camera 121 processes image frames such as a still image or a video obtained by an image sensor in a video call mode or a capturing mode. The processed image frame may be displayed on the display unit 151 or stored in the memory 170.

The microphone 122 processes external sound signals as electrical speech data. The processed speech data may be utilized variously according to a function (or an application program being executed) being performed in the AI apparatus 100. Moreover, various noise canceling algorithms for removing noise occurring during the reception of external sound signals may be implemented in the microphone 122.

The user input unit 123 is to receive information from a user and when information is inputted via the user input unit 123, the processor 180 may control an operation of the AI apparatus 100 to correspond to the inputted information.

The user input unit 123 may include a mechanical input means (or a mechanical key, for example, a button, a dome switch, a jog wheel, and a jog switch at the front, back or side of the AI apparatus 100) and a touch type input means. As one example, a touch type input means may include a virtual key, a soft key, or a visual key, which is displayed on a touch screen through software processing or may include a touch key disposed at a portion other than the touch screen.

The sensing unit 140 may also be referred to as a sensor unit.

The sensing unit 140 may include at least one of an electrostatic sensor, a pressure sensor, or a piezoelectric sensor disposed at a portion where the user contacts, and the sensor data for at least one of the contact surface and the intensity of contact when the user contacts the massage chair can be obtained. In this case, the processor 180 may obtain information about at least one of body shape, a posture, or a position of the user based on the sensor data obtained by the sensing unit 140.

The sensor included in the sensing unit 140 is not limited to the above-described electrostatic sensor, pressure sensor, and piezoelectric sensor, and may be any sensor capable of collecting sensor data that may be used to obtain information about at least one of a body shape, posture, and position of the user, such as an inertial sensor, a magnetic sensor, a gravity sensor, a gyroscope sensor, an acceleration sensor, an ultrasonic sensor, an optical sensor, or the like.

The output unit 150 may include at least one of a display unit 151, a sound output module 152, a haptic module 153, or an optical output module 154.

The display unit 151 may display (output) information processed in the AI apparatus 100. For example, the display unit 151 may display execution screen information of an application program running on the AI apparatus 100 or user interface (UI) and graphic user interface (GUI) information according to such execution screen information.

The display unit 151 may be formed with a mutual layer structure with a touch sensor or formed integrally, so that a touch screen may be implemented. Such a touch screen may serve as the user input unit 123 providing an input interface between the AI apparatus 100 and a user, and an output interface between the AI apparatus 100 and a user at the same time.

The sound output module 152 may output audio data received from the wireless communication unit 110 or stored in the memory 170 in a call signal reception or call mode, a recording mode, a speech recognition mode, or a broadcast reception mode.

The sound output module 152 may include a receiver, a speaker, and a buzzer.

The haptic module 153 generates various haptic effects that a user can feel. A representative example of a haptic effect that the haptic module 153 generates is vibration.

The optical output module 154 outputs a signal for notifying event occurrence by using light of a light source of the AI apparatus 100. An example of an event occurring in the AI apparatus 100 includes message reception, call signal reception, missed calls, alarm, schedule notification, e-mail reception, and information reception through an application.

The massage unit 160 is configured to perform massage by being in contact with the user, and may include at least one of a head massage unit 161 supporting the head of the user, a back massage unit 162 supporting the back of the user, an arm massage unit 163 supporting the arm of the user, a hip massage unit 164 supporting the hip of the user, or a leg massage unit 165 supporting the leg of the user. Each of the head massage unit 161, the back massage unit 162, the arm massage unit 163, the hip massage unit 164, and the leg massage unit 165 may include an airbag.

The driving unit 190 may generate a physical movement or physical force for massage through the motor 191 generating a rotational force and may transmit the generated physical movement or physical force to the massage unit 160. For example, the driving unit 190 may move the massage head using the physical force generated by the motor 191 and may massage the body of the user through the movement of the massage head. The massage head may be viewed as a configuration of the driving unit 190 or may be viewed as a configuration of the massage unit 160.

The driving unit 190 may adjust the air pressure of the airbag or the air injection amount into the airbag through an inflator 192 for injecting air into the airbag of the massage unit 160. In other words, the inflator 192 may attempt to supply air to the airbag according to the set supply air pressure, if the air pressure of the airbag is higher than the supply air pressure, the airbag deflates until the air pressure of the airbag is equal to the supply air pressure, and if the air pressure of the airbag is lower than the supply air pressure, the airbag is inflated until the air pressure of the airbag is equal to the supply air pressure.

In particular, the driving unit 190 may adjust the massage intensity by adjusting the rotational speed of the motor 191 or the air pressure or air injection amount of the inflator 192. For example, the driving unit 190 may increase the massage intensity by increasing the rotational speed of the motor 191 or reducing the air pressure of the inflator 192. On the contrary, the driving unit 190 may reduce the massage intensity by reducing the rotational speed of the motor 191 or increasing the air pressure of the inflator 192. Since the airbag is inflated as the air pressure of the airbag increases, the distance between the body of the user and the massage head corresponding to the airbag increases, so that the massage intensity of the body part corresponding to the airbag decreases.

In FIG. 4, the driving unit 190 is illustrated in a configuration separated from the massage unit 160, but the present disclosure is not limited thereto. In an embodiment, each of the head massage unit 161, the back massage unit 162, the arm massage unit 163, the hip massage unit 164, and the leg massage unit 165 included in the massage unit 160 may individually include at least one driving unit 190.

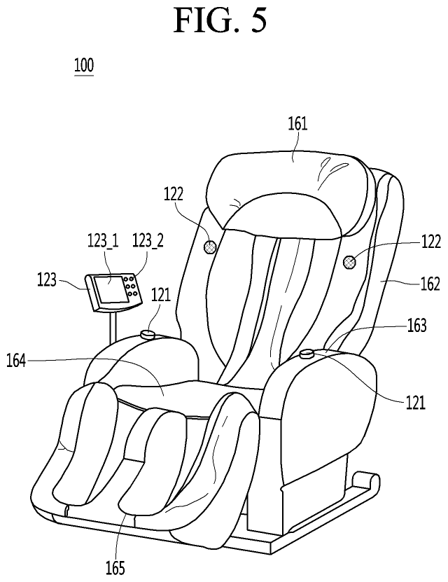

FIG. 5 is a perspective view illustrating an artificial intelligence apparatus 100 according to an embodiment of the present disclosure.

Referring to FIG. 5, the artificial intelligence apparatus 100 or the artificial intelligence massage apparatus 100 may be a massage apparatus in the form of a chair. The massage apparatus in the form of a chair may include not only a general massage chair but also a chair having a massage unit 160, a driving unit 190, and the like, which may provide a massage function or a massage operation.

In one embodiment, the artificial intelligence apparatus 100 may be a car seat including a massage unit 160 and a driving unit 190 to provide a massage function. If the artificial intelligence apparatus 100 is a car seat, the artificial intelligence apparatus 100 may be regarded as a part of the vehicle on which the artificial intelligence apparatus is mounted. For safety, the artificial intelligence apparatus 100 may operate only in a situation in which the vehicle is self-driving.

The artificial intelligence apparatus 100 may include at least one of a head massage unit 161 supporting the head of the user, a back massage unit 162 supporting the back of the user, an arm massage unit 163 supporting the arm of the user, a hip massage unit 164 supporting the hip of the user, or a leg massage unit 165 supporting the leg of the user.

Each of the head massage unit 161, the back massage unit 162, the arm massage unit 163, the hip massage unit 164, and the leg massage unit 165 may include one or more massage heads, and, by operating by physical movement or physical force generated from the motor 191 of the driving unit 190, each massage head can massage at least a part of the body of the user. Each massage head may include one or more massage rods.

Meanwhile, each of the head massage unit 161, the back massage unit 162, the arm massage unit 163, the hip massage unit 164, and the leg massage unit 165 may include at least one lower massager. For example, the head massage unit 161 may include at least one of a head massager that can massage the head of the user and a neck massager that can massage the neck of the user. The back massage unit 162 may include at least one of a shoulder massager that can massage the shoulder of the user, a back massager that can massage the back of the user, and a waist massager that can massage the waist of the user. The leg massage unit 165 may include at least one of a thigh massager that can massage the thigh of the user, a calf massager that can massage the calf of the user, and a foot massager that can massage the foot of the user.

In addition, the artificial intelligence apparatus 100 may include a user input unit 123 or a user interface unit. The user input unit 123 may include a display unit 123_1 that displays information under the control of the processor 180, and an input unit 123_2 that receives an input from a user and transmits the input to the processor 180. The display unit 123_1 included in the user input unit 123 may refer to the display unit 151 of the output unit 150.

In addition, the artificial intelligence apparatus 100 may include at least one camera 121 and at least one microphone 122. The camera 121 may be installed at a position capable of obtaining image data including a face of a user using the artificial intelligence apparatus 100. The microphone 122 may be installed at a position capable of receiving the utterance speech of the user.

In an embodiment, in a situation in which the user looks at the front, the artificial intelligence apparatus 100 may use the camera 121 and the microphone 122 installed in the front direction to obtain image data including the face of the user and speech data including the utterance speech of the user. In the situation where the user looks at the side, the artificial intelligence apparatus 100 may use the camera 121 and the microphone 122 installed in the side direction to obtain image data including the face of the user and speech data including the utterance speech of the user.

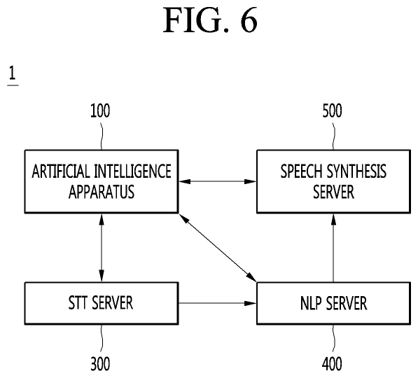

FIG. 6 is a block diagram illustrating an AI system 1 according to an embodiment of the present disclosure.

Referring to FIG. 6, the AI system 1 may include an AI apparatus 100, a speech-to-text (STT) server 300, a natural language processing (NLP) server 400 and a speech synthesis server 500.

The AI apparatus 100 may transmit speech data to the STT server 300. The STT server 300 may convert the speech data received from the AI apparatus 100 into text data. The NLP server 400 may receive text data from the STT server 300. The NLP server 400 may analyze the intent of the text data based on the received text data. The NLP server 400 may transmit intent analysis information indicating the result of analyzing the intent to the AI apparatus 100 or the speech synthesis server 500. The speech synthesis server 500 may generate a synthesis speech reflecting the intent of the user based on the intent analysis information and transmit the generated synthesis speech to the AI apparatus 100.

The STT server 300 may increase accuracy of speech-to-text conversion using a language model. The language model may mean a model capable of calculating a probability of a sentence or calculating a probability of outputting a next word when previous words are given. For example, the language model may include probabilistic language models such as a unigram model, a bigram model and an N-gram model. The unigram is a model that assumes that all words are completely independent of each other and calculates a probability of a word sequence as a product of probabilities of words. The bigram model is a model that assumes that use of a word depends on only one previous word. The N-gram model is a model that assume that use of a word depends on previous (n-1) words.

In other words, the STT server 300 may determine whether the converted text data is appropriately converted from the speech data using a language model, thereby increasing accuracy of conversion from the speech data into the text data.

The NLP server 400 may sequentially perform a morpheme analysis step, a syntax analysis step, a speech-act analysis step, an interaction processing step with respect to text data, thereby generating intent analysis information.

The morpheme analysis step refers to a step of classifying the text data corresponding to the speech uttered by the user into morphemes as a smallest unit having a meaning and determining the part of speech of each of the classified morphemes. The syntax analysis step refers to a step of classifying the text data into a noun phrase, a verb phrase, an adjective phrase, etc. using the result of the morpheme analysis step and determining a relation between the classified phrases. Through the syntax analysis step, the subject, object and modifier of the speech uttered by the user may be determined. The speech-act analysis step refers to a step of analyzing the intent of the speech uttered by the user using the result of the syntax analysis step. Specifically, the speech-act step refers to a step of determining the intent of a sentence such as whether the user asks a question, makes a request, or expresses simple emotion. The interaction processing step refers to a step of determining whether to answer the user's utterance, respond to the user's utterance or question about more information, using the result of the speech-act step.

The NLP server 400 may generate intent analysis information including at least one of the answer to, a response to, or a question about more information on the intent of the user's utterance, after the interaction processing step.

Meanwhile, the NLP server 400 may receive the text data from the AI apparatus 100. For example, when the AI apparatus 100 supports the speech-to-text conversion function, the AI apparatus 100 may convert the speech data into the text data and transmit the converted text data to the NLP server 400.

The speech synthesis server 500 may synthesize prestored speech data to generate a synthesized speech. The speech synthesis server 500 may record the speech of the user selected as a model and divide the recorded speech into syllables or words. The speech synthesis server 500 may store the divided speech in an internal or external database in syllable or word units.

The speech synthesis server 500 may retrieve syllables or words corresponding to the given text data from the database and synthesize the retrieved syllables or words, thereby generating the synthesized speech.

The speech synthesis server 500 may store a plurality of speech language groups respectively corresponding to a plurality of languages. For example, the speech synthesis server 500 may include a first speech language group recorded in Korean and a second speech language group recorded in English.

The speech synthesis server 500 may translate text data of a first language into text of a second language and generate a synthesized speech corresponding to the translated text of the second language using the second speech language group.

The AI system 1 may further include an AI server 200. The AI server 200 may learn at least one of an STT engine used in the STT server 300, an NLP engine used in the NLP server 400 or a speech synthesis engine used in the speech synthesis server 500. In other words, at least one of the STT server 300, the NLP server 400 or the speech synthesis server 500 may use models or engines trained in the AI server 200.

Although the AI apparatus 100, the STT server 300, the NLP server 400 and the speech synthesis server 500 are shown as being divided in FIG. 5, the present disclosure is not limited thereto. In one embodiment, some of the AI server 200, the STT server 300, the NLP server 400 or the speech synthesis server 500 may be configured as one server. In one embodiment, some of the STT server 300, the NLP server 400 or the speech synthesis server 500 may be included in the AI apparatus 100. This means that the AI apparatus 100 performs the function of the STT server 300, the NLP server 400 or the speech synthesis server 500.

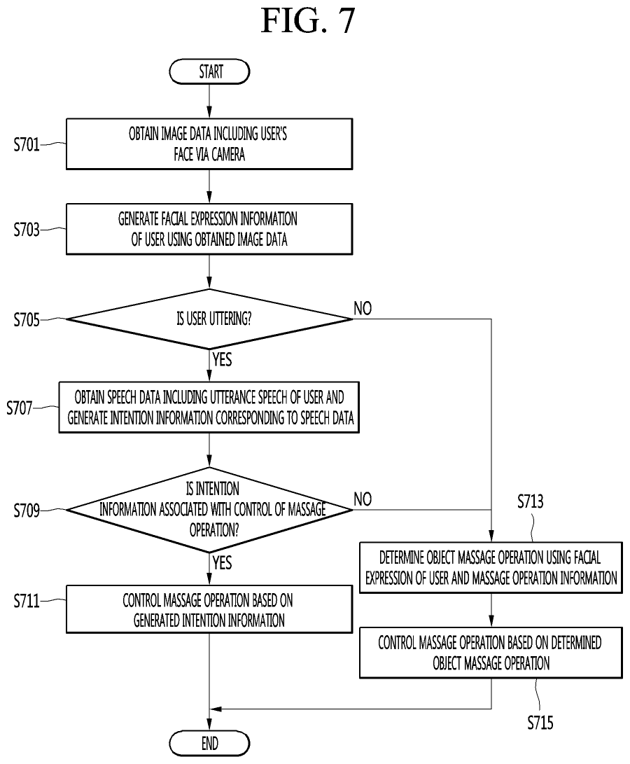

FIG. 7 is a flowchart illustrating a method for controlling a massage operation in consideration of a facial expression or utterance of a user according to an embodiment of the present disclosure.

Referring to FIG. 7, the processor 180 of the artificial intelligence massage apparatus 100 obtains image data including a face of a user via the camera 121 (S701).

The artificial intelligence massage apparatus 100 may include at least one or more cameras 121, and the processor 180 may use the camera that is closest to a viewing direction of the face of the user using the artificial intelligence massage apparatus 100 and thus may obtain image data including the face of the user. If the user is looking at the front, the processor 180 may obtain image data using the camera 121 located at the front.

The processor 180 may obtain image data from each camera 121 and determine the face of the user and a face direction of the user from each image data. The processor 180 may select and use the image data closest to the center of the face direction determined from the image data. For example, if the artificial intelligence massage apparatus 100 includes a first camera on the front, a second camera on the left side, and a third camera on the right side, and if the user faces the front, the face direction of the user is close to the center in the first image data obtained by the first camera, but the face direction of the user is far away from the center in the second image data obtained by the second camera and the third image data obtained by the third camera. In this case, the processor 180 may select and use only the first image data without using the second image data and the third image data.

In addition, the processor 180 of the artificial intelligence massage apparatus 100 generates the user's facial expression information by using the obtained image data (S703).

In an embodiment, the user's facial expression information may include scores for predetermined facial expression items. For example, the processor 180 may generate the user's facial expression information by determining a score for each of facial expression items such as neutral, frown, and smile.

In an embodiment, the user's facial expression information may include scores for predetermined emotion items. For example, the processor 180 may generate the user's facial expression information by determining scores for emotion items such as anger, a contempt, a disgust, a fear, a happiness, a neutral, a sadness, and a surprise from the face or the facial expression of the user.

The processor 180 may generate the user's facial expression information from the obtained image data by using the facial expression information generation model stored in the memory 170. Alternatively, the processor 180 transmits the obtained image data to the artificial intelligence server 200 via the communication unit 110, and if the processor 260 of the artificial intelligence server 200 uses the facial expression information generation model stored in the memory 230 and thus generates the user's facial expression information from the obtained image data, the processor 180 may receive the user's facial expression information generated from the artificial intelligence server 200 via the communication unit 110.

When the image data is input, the facial expression information generation model may be a model outputting a score for each facial expression item corresponding to the face of the user included in the input image data, or a model outputting a score for each emotion item corresponding to the face of the user. The facial expression information generation model includes an artificial neural network and may be a model that is trained using a machine learning algorithm or a deep learning algorithm.

The learning processor 130 of the artificial intelligence massage apparatus 100 or the learning processor 240 of the artificial intelligence server 200 may train the facial expression information generation model using the first training data. The first training data may include image data including the face of the user and a score for each facial expression item corresponding to the face of the user or a score for each emotion item. The score for each facial expression item corresponding to the face of the user or the score for each emotion item is a label or label data for the corresponding image data and may can function as the result or correct answer to be inferred from the image data by the facial expression information generation model.

In addition, the processor 180 of the artificial intelligence massage apparatus 100 determines whether the user is uttering a sound or voice input using the obtained image data (S705).

The processor 180 may determine whether the user is uttering a sound or voice input by detecting the lips of the user from the image data and determining whether the lips of the user are moving. If the user uses the artificial intelligence massage apparatus 100, the user is at a fixed position with respect to the artificial intelligence massage apparatus 100. Therefore, the face of the user may be clearly included in the image data obtained using the camera 121, and thus, the lips of the user may be detected with high accuracy and the movement of the lips may be determined.

If the user is uttering as a result of the determination in step S705, the processor 180 of the artificial intelligence massage apparatus 100 obtains speech data including the utterance speech of the user, and generates intention information that corresponds to the obtained speech data (S707).

Typically, the speech recognition function operates when the user utters a predetermined starting word (or a predetermined wakeup word). However, in the present disclosure, it is possible to determine whether the user is uttering based on the movement of the lips of the user, so that speech data may be obtained to recognize the utterance speech of the user without a separate starting word.

Obtaining the speech data in order to recognize the utterance speech includes selecting only the speech data of a situation where it is determined that the user is uttering while continuously obtaining the speech data via the microphone 122 constantly activated, or obtaining the speech data by activating the microphone 122 only in a situation where it is determined that the user is uttering.

The utterance speech of the user may be a speech related to the massage of the artificial intelligence massage apparatus 100, or may be a normal conversation speech irrelevant to the massage, and may be a sound such as humming and moaning indicating satisfaction or pain. In the present disclosure, sounds such as humming and moaning can also be regarded as part of speech.

The processor 180 may obtain speech data via the microphone 122 or may receive speech data from an external device via the communication unit 110.

The speech data is obtained by converting a sound wave including the speech of the user into a digital signal. For example, the speech data may be an audio file in various formats such as pulse code modulation (PCM), way, mp3, and the like.

The processor 180 may remove noise from speech data obtained by pre-processing. The processor 180 may generate speech data from which noise is removed by using a noise removing engine or a noise removing filter directly, or transmit speech data to the artificial intelligence server 200 and receive speech data from which noise is removed. In addition, the volume of the speech data may be adjusted to a predetermined level. Adjusting the volume of speech data can also be seen as part of the preprocessing process. Hereinafter, the speech data may refer to speech data from which noise is removed through preprocessing.

The processor 180 may convert speech data into text using the STT engine, and generate intention information corresponding to the converted text using the NLP engine. When converting the speech data into text, the processor 180 can calculate a word-specific probability corresponding to each section of the speech data, convert the speech data into text based on the calculated probability, and generate intention information based on the converted text. In particular, the processor 180 may convert the speech data into text by combining words having the highest probability.

Alternatively, the processor 180 may generate intention information corresponding to speech data using the STT server 300 and the NLP server 400. For example, the processor 180 may transmit speech data to the STT server 300 via the communication unit 110, the STT server 300 may convert the received speech data into text and transmit the converted text to the NLP server 400, the NLP server 400 may generate intention information corresponding to the received text and transmit the generated intention information to the artificial intelligence apparatus 100.

In an embodiment, the processor 180 can determine an actual facial direction of the user based on the position of the camera 121 obtaining the image data and the facial direction of the user in the image data, adjust the beamforming direction of the microphone 122 based on the determined actual facial direction of the user, and obtain speech data including the utterance speech of the user via the microphone 122. The artificial intelligence massage apparatus 100 may include a plurality of microphones 122 or a microphone 122 composed of a microphone array, and the processor 180 may set the beamforming direction using at least some of the plurality of microphones or the microphone array and may obtain speech data in the beamforming direction using the microphone 122 in which the beamforming direction is set.

In addition, the processor 180 of the artificial intelligence massage apparatus 100 determines whether the generated intention information is related to the control of the massage operation (S709).

When the intention information includes the control of the massage operation, the processor 180 may determine that the intention information is related to the control of the massage operation. Specifically, the control of the massage operation may include the setting of a massage course, setting of massage time, setting of massage intensity, setting of a massage part (object), and the like.

The control of the massage operation may mean a control of a massage course or a massage schedule and may include a sequential setting of a massage part, a massage time, massage intensity, and the like. The massage course may be selected from at least one preset massage course, but may be selected from a user set massage course in which at least a part of the preset massage course is modified by the input of the user.

The setting of the massage part (target) may include not only the setting whether to operate each of the head massage unit 161, the back massage unit 162, the arm massage unit 163, the hip massage unit 164, and the leg massage unit 165 included in the massage unit 160, but also the setting of the position of the massage head in a single massage unit.

The control of the massage operation may include not only control for setting the massage course itself but also control for setting individual items such as a massage part, a massage time, and a massage intensity constituting the massage course. For example, the control of the massage operation may include not only a control for changing the first preset massage course into a second preset massage course in a situation in which it is currently operating as the first preset massage course, but also a control for generating the user set massage course by changing only the massage intensity (or the massage time, massage part, or the like) corresponding to the current massage operation among the first preset massage course and modifying a portion of contents of the first preset massage course and for changing the first preset massage course into the generated user set massage course.

For example, if the utterance speech of the user is “make it stronger,” processor 180 may generate intent information, such as “increase massage intensity.” If the utterance speech of the user is “massage the leg,” the processor 180 may generate intention information such as “operate leg massage unit” or “start leg massage”. If the utterance speech of the user is “Now there is good. Give me more massage there”, the processor 180 may generate intention information such as “fixed massage position and increase massage time at fixed position”. The above examples represent intention information related to control of the massage operation.

For example, if the utterance speech of the user is “very good,”” the processor 180 may generate intention information such as “positive feedback” or “satisfaction”. If the utterance speech of the user is “not so good,” the processor 180 may generate intent information such as “negative feedback” or “dissatisfaction”. The above examples represent intention information not related to the control of the massage operation.

If the intention information is related to the control of the massage operation as a result of the determination in step S709, the processor 180 of the artificial intelligence massage apparatus 100 controls the massage operation based on the generated intention information (S711).

For example, if the intention information is “increase in massage intensity”, the processor 180 may increase the intensity of a massage currently being performed. If the intention information is “leg massage unit operation” or “leg massage start”, the processor 180 may operate the leg massage unit 165. If the intention information is “massage position fixation and increase in massage time at a fixed position”, the processor 180 can fix the position of the massage head in the massage unit 160 currently operating, and increase the massage time at the fixed position.

The processor 180 can output a response (or feedback) to the control of the massage operation to the user through the output unit 150 or transmit an output signal for outputting a response to the control of the massage operation to a user terminal via the communication unit 110. For example, if the processor 180 performs the control to increase the massage intensity, the processor 180 may output a notification, a message, or the like indicating that the massage intensity is increased through the output unit 150 or a user terminal.

The processor 180 may generate a response speech for outputting response information and output the response speech through the sound output unit 152. In detail, the processor 180 can generate a response sentence using a Natural Language Generation (NLG) technique, convert the response sentence generated using a text to speech (TTS) engine into a response speech, and outputs a sound, and output through the response speech converted through the sound output unit 152.

In an embodiment of the present disclosure, the processor 180 may generate training data for use in training the first massage operation determination model or the second massage operation determination model, which will be described later, using the control information of the massage operation based on the generated intention information. The processor 180 may generate training data including the user's facial expression information, massage operation information of the artificial intelligence massage apparatus 100, and control information of the massage operation, or the processor 180 may generate training data including image data including the face of the user, massage operation information of the artificial intelligence massage apparatus 100, and the control information of the massage operation. The former training data may be used as second training data to be described later, and the latter training data may be used as third training data to be described later.

If the user is not uttering as a result of determination in step 5705, or if the intention information is not related to the control of the massage operation as a result of the determination in step S709, the processor 180 of the artificial intelligence massage apparatus 100 determines an object massage operation using the user's facial expression information and the massage operation information (S713).

The object massage operation may mean a massage operation that is determined to be appropriate from the massage operation information of the current artificial intelligence massage apparatus 100 and the facial expression of the user. For example, if the current massage operation is proceeding at the maximum intensity and the user is sick and frowning, the object massage operation may be a massage operation with low massage intensity.

The processor 180 uses the first massage operation determination model stored in the memory 170 to determine an object massage operation from the user's facial expression information generated from the obtained image data and massage operation information of the current artificial intelligence massage apparatus 100. Alternatively, the processor 180 transmits the user's facial expression information and the massage operation information to the artificial intelligence server 200 via the communication unit 110, and when the processor 260 of the artificial intelligence server 200 determines an object massage operation from the user's facial expression information and the massage operation information using the first massage operation determination model stored in the memory 230, the processor 180 can receive the object massage operation determined from the artificial intelligence server 200 via the communication unit 110.

The first massage operation determination model may be a model for outputting an object massage operation suitable for the user's facial expression information and the massage operation information when the user's facial expression information and the massage operation information are input. The first massage operation determination model includes an artificial neural network and may be a model that is trained using a machine learning algorithm or a deep learning algorithm.

The learning processor 130 of the artificial intelligence massage apparatus 100 or the learning processor 240 of the artificial intelligence server 200 may learn the first massage operation determination model using the second training data. The second training data may include the user's facial expression information, massage operation information, and object massage operation (or object massage operation information). The object massage operation is a label for the user's facial expression information and the massage operation information, and may function as a result or a correct answer to be inferred from the user's facial expression information and the massage operation information by the first massage operation determination model.

The first massage operation determination model may refer to a model for determining an object massage operation by using the user's facial expression information generated from image data including a face of a user. However, the present disclosure is not limited thereto. In other words, in one embodiment, the processor 180 may determine the object massage operation from the image data and the massage operation information using the second massage operation determination model. In this case, the processor 180 may determine the object massage operation from image data including the face of the user and current massage operation information without generating the user's facial expression information. An embodiment using the second massage operation determination model will be described later.

In one embodiment, the first massage operation determination model does not use only image data of a single frame corresponding to a specific time point (for example, the current time point), and can use image data of a plurality of frames of a predetermined interval before and after a specific time point. For example, the first massage operation determination model may be a model for determining an object massage operation based on facial expression information and massage operation information generated from image data for n seconds before and after a specific time point. If the object massage operation is determined in real-time, the first massage operation determination model may determine the object massage operation based on the facial expression information and the massage operation information generated from the image data for the previous n seconds with respect to the current time point.

In addition, the processor 180 of the artificial intelligence massage apparatus 100 controls the massage operation based on the determined object massage operation (S715).

If the current massage operation and the determined object massage operation are the same, the processor 180 may maintain the current massage operation in progress. On the other hand, if the current massage operation is different from the determined object massage operation, the processor 180 may control the massage operation according to the object massage operation.

FIG. 7 discloses a method for controlling a massage operation in consideration of the face of the user and the utterance speech of the user, but the present disclosure is not limited thereto. In other words, the utterance speech of the user is only one example of an interaction method for controlling the massage operation, and if the user inputs a command for controlling the massage operation via the user input unit 123 such as a remote controller, the artificial intelligence massage apparatus 100 may control the massage operation based on a user input. In this case, the processor 180 may generate training data for use in training the first massage operation determination model or the second massage operation determination model based on the control information of the massage operation based on the input of the user.

In addition, although FIG. 7 illustrates that the step S703 of generating the user's facial expression information is performed immediately after the step S701 of obtaining of the image data, the present disclosure is not limited thereto. In other words, in an embodiment, the step S703 of generating of the user's facial expression information may be performed immediately before the step S713 of determining of the object massage operation.

The steps illustrated in FIG. 7 may be repeatedly performed, and accordingly, the artificial intelligence massage apparatus 100 may continuously control the massage operation.

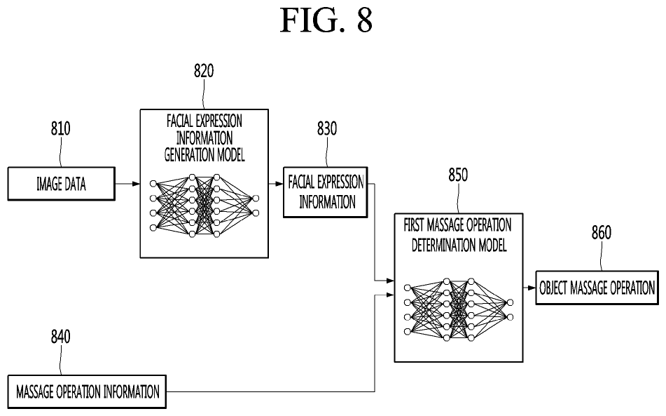

FIG. 8 is a view illustrating a method for determining an object massage operation by using a first massage operation determination model according to an embodiment of the present disclosure.

Referring to FIG. 8, the processor 180 obtains image data 810 including a face of a user via the camera 121 and inputs the obtained image data 810 into the facial expression information generation model 820 to generate the user's facial expression information 830. The structure of the facial expression information generation model illustrated in FIG. 8 is merely an example, and the present disclosure is not limited thereto. In one embodiment, the facial expression information generation model may include a convolutional neural network (CNN).

In addition, the processor 180 obtains the massage operation information 840 of the current time point, determines the object massage operation 860 by inputting the generated facial expression information 830 and the obtained massage operation information 840 to the first massage operation determination model 850, and then. Similarly, the structure of the first massage operation determination model 850 illustrated in FIG. 8 is just one example, and the present disclosure is not limited thereto. In an embodiment, the first massage operation determination model 850 may include a recurrent neural network (RNN) to determine the object massage operation 860 from data input in time series.

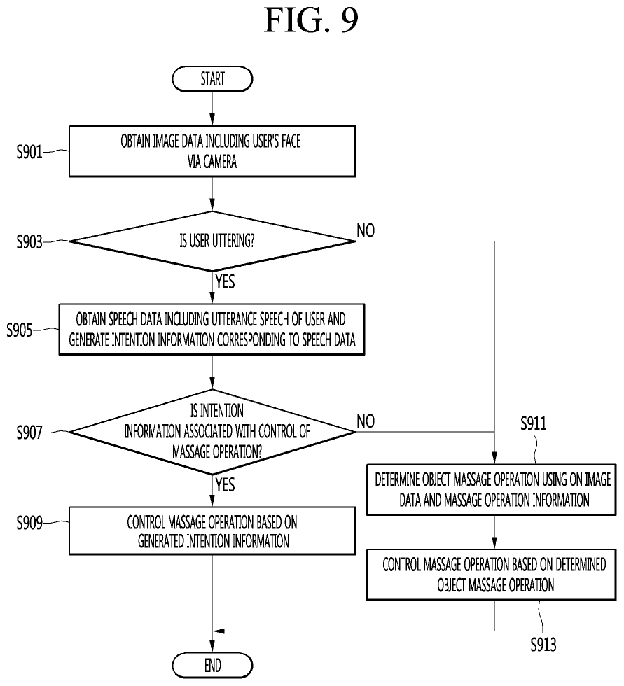

FIG. 9 is a flowchart illustrating a method for controlling a massage operation in consideration of a facial expression or utterance of a user according to an exemplary embodiment of the present disclosure.

Referring to FIG. 9, the processor 180 of the artificial intelligence massage apparatus 100 obtains image data including a face of a user via the camera 121 (S901). This corresponds to the step S701 of FIG. 7 of obtaining the image data.

In addition, the processor 180 of the artificial intelligence massage apparatus 100 determines whether the user is uttering using the obtained image data (S903). This corresponds to the step S705 of FIG. 7 of determining whether the user is uttering.

If the user is uttering as a result of the determination in step S903, the processor 180 of the artificial intelligence massage apparatus 100 obtains speech data including the utterance speech of the user and generates intention information corresponds to the obtained speech data (S905). This corresponds to the step S707 of FIG. 7 of generating intention information corresponding to the speech data.

In addition, the processor 180 of the artificial intelligence massage apparatus 100 determines whether the generated intention information is related to the control of the massage operation (S907). This corresponds to the step S709 of FIG. 7 of determining whether the intention information is related to the control of the massage operation.

If the intention information is related to the control of the massage operation as a result of the determination in step S907, the processor 180 of the artificial intelligence massage apparatus 100 controls the massage operation based on the generated intention information (S909). This corresponds to the step S711 of FIG. 7 of controlling the massage operation based on the generated intention information.

In an embodiment of the present disclosure, the processor 180 may generate training data for use in training a second massage operation determination model, which will be described later, using control information of the massage operation based on the generated intention information. The processor 180 may generate training data including image data including a face of a user, massage operation information of the artificial intelligence massage apparatus 100, and control information of the massage operation. Such training data may be used as third training data described later.

If the user is not uttering as a result of the determination in step S903, or if the intention information is not related to the control of the massage operation as a result of the determination in step S907, the processor 180 of the artificial intelligence massage apparatus 100 determines the object massage operation using the massage operation information (S911).

The object massage operation may mean a massage operation that is determined to be appropriate from the massage operation information of the current artificial intelligence massage apparatus 100 and the facial expression of the user. For example, if the current massage operation is proceeding at the maximum intensity and the user is sick and frowning, the object massage operation may be a massage operation with low massage intensity.

The processor 180 may determine the object massage operation from the obtained image data and massage operation information of the current artificial intelligence massage apparatus 100 using the second massage operation determination model stored in the memory 170. Alternatively, the processor 180 can transmit image data and massage operation information to the artificial intelligence server 200 via the communication unit 110, and when the processor 260 of the artificial intelligence server 200 determines the object massage operation from the image data and the massage operation information using the second massage operation determination model stored in the memory 230, the processor 180 can receive the object massage operation determined from the artificial intelligence server 200 via the communication unit 110.

The second massage operation determination model may be a model for outputting an object massage operation suitable for the image data and the massage operation information, when the image data including the face of the user and the massage operation information are input. In other words, the second massage operation model may refer to a model for directly determining an object massage operation from image data without generating the user's facial expression information by end-to-end learning. The second massage operation determination model includes an artificial neural network and may be a model that is trained using a machine learning algorithm or a deep learning algorithm.

The learning processor 130 of the artificial intelligence massage apparatus 100 or the learning processor 240 of the artificial intelligence server 200 may train the second massage operation determination model using the third training data. The third training data may include image data including the face of the user, massage operation information, and object massage operation (or object massage operation information). The object massage operation is a label for the image data and the massage operation information, and may function as a result or a correct answer to be inferred from the user's facial expression information and the massage operation information by the massage operation determination model.

In one embodiment, the second massage operation determination model does use not only image data oa a single frame corresponding to a specific time point (for example, the current time point), but also image data of a plurality of frames of a predetermined interval before and after a specific time point. For example, the second massage operation determination model may be a model for determining an object massage operation based on image data and massage operation information for n seconds before and after the specific time point. If the object massage operation is determined in real-time, the second massage operation determination model may determine the object massage operation based on image data and massage operation information for the previous n seconds based on the current time point.

In addition, the processor 180 of the artificial intelligence massage apparatus 100 controls the massage operation based on the determined object massage operation (S913). This corresponds to the step S715 of FIG. 7 of controlling the massage operation based on the object massage operation.

The steps illustrated in FIG. 9 may be repeatedly performed, and accordingly, the artificial intelligence massage apparatus 100 may continuously control the massage operation.

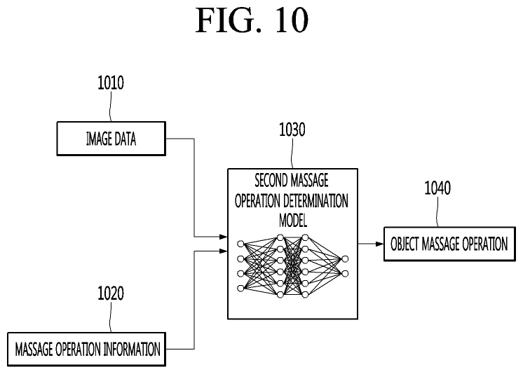

FIG. 10 is a view illustrating a method of determining an object massage operation using a second massage gesture determination model according to an exemplary embodiment of the present disclosure.

Referring to FIG. 10, the processor 180 obtains image data 1010 including the face of the user via the camera 121, obtains massage operation information 1020 at the present time, and inputs the obtained image data 1010 and the obtained massage operation information 1020 to the second massage operation determination model 1030 to determine the object massage operation 1040. Similarly, the structure of the second massage operation determination model 1030 illustrated in FIG. 10 is merely an example, and the present disclosure is not limited thereto. In an embodiment, the second massage operation determination model 1030 may include a recurrent neural network (RNN) to determine the object massage operation 1040 from data input in time series.

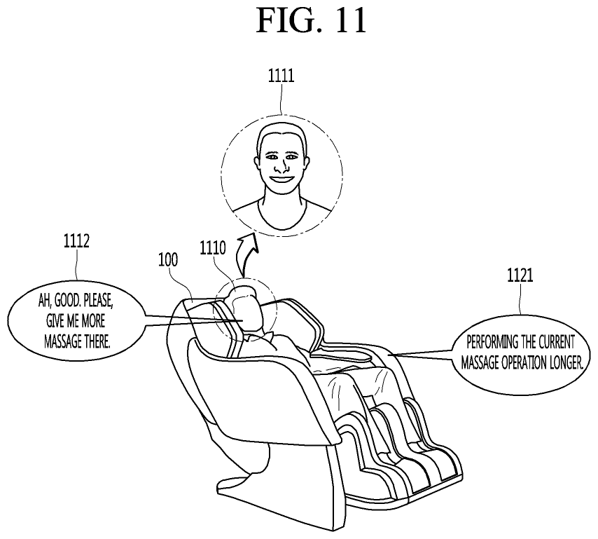

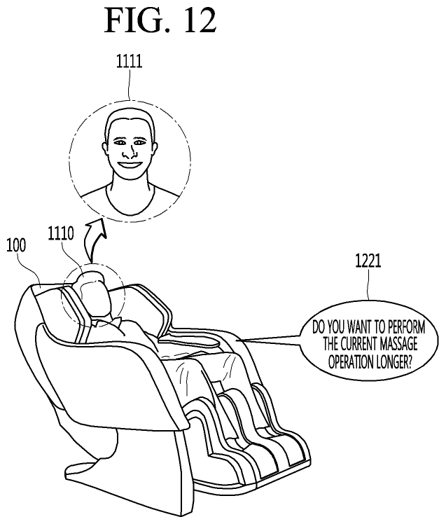

FIGS. 11 and 12 are views illustrating examples of controlling a massage operation in consideration of the facial expression or utterance of the user according to an embodiment of the present disclosure.

Referring to FIGS. 11 and 12, the user 1110 may receive a massage according to a preset massage operation or a massage operation set by the user 1110 using the artificial intelligence massage apparatus 100.

If the user 1110 is satisfied with the present massage operation and makes a smiley expression 1111 or a satisfied expression, and is uttering speech such as “Oh, good. Please, give me more massage there.” 1112, the artificial intelligence massage apparatus 100 may determine that the massage operation currently being performed is satisfactory to the user 1110 based on the utterance speech 1112 of the user 1110. In addition, the artificial intelligence massage apparatus 100 may further increase the operation time of the current massage operation and output a response speech such as “Performing the current massage operation longer.” 1121. Alternatively, even if the user 1110 is not directly uttering the speech but the user inputs to increase the operation time of the current massage operation via the user input unit 123 such as a remote controller, the artificial intelligence massage apparatus 100 may extend the operation time of the current massage operation and, if necessary, output a response speech such as “Performing the current massage operation longer.” 1121.

The artificial intelligence massage apparatus 100 can generate training data for use in the learning of the massage operation determination model based on current massage operation information, the user's facial expression information (or image data including the face), the massage operation control the information of the user 1110. In addition, the massage operation determination model may be re-learned or updated using the generated training data. The artificial intelligence massage apparatus 100 may regard the massage operation controlled by the user 1110 being uttering as a speech or the massage operation controlled via the user input unit 123 by the user 1110 as an object massage operation.

Therefore, if the artificial intelligence massage apparatus 100 performs the same massage operation as the massage operation in the example of FIG. 11 later, and the user 1110 makes a smiley expression 1111 or a satisfactory expression, the artificial intelligence massage apparatus 100 can output a query speech such as “Do you want to perform the current massage operation longer?” 1221 to further increase the operation time of the current massage operation, even though there is no utterance or control input of the user 1110. If the user 1110 utters and agrees, the artificial intelligence massage apparatus 1110 may further increase the operation time of the current massage operation.

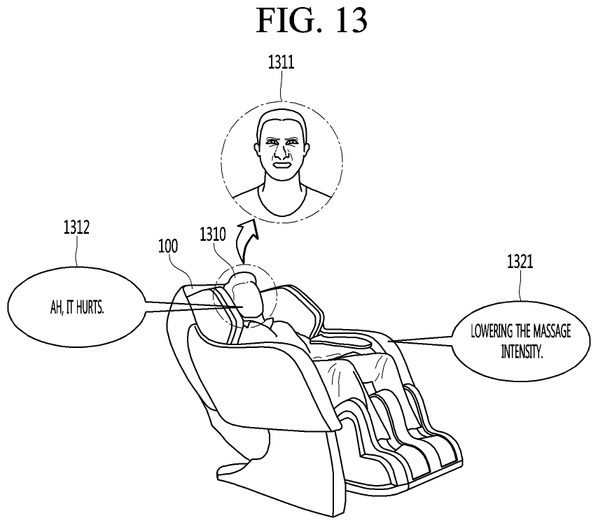

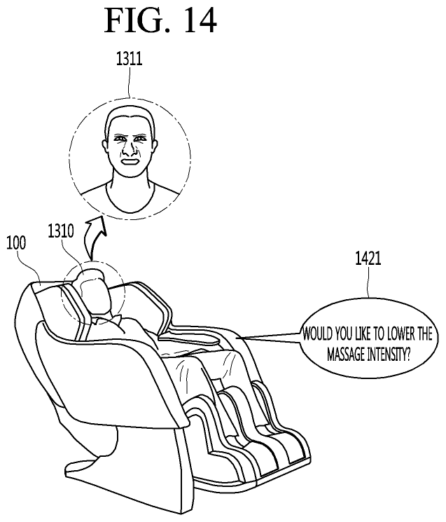

FIGS. 13 and 14 are views illustrating examples of controlling a massage operation in consideration of the facial expression or utterance of the user according to an embodiment of the present disclosure.

Referring to FIGS. 13 and 14, the user 1310 may receive a massage according to a preset massage operation or a massage operation set by the user 1310 using the artificial intelligence massage apparatus 100.

If the user 1310 has a painful expression 1311 or a frowning expression and is uttering speech such as “Oh, it hurts” 1312, the artificial intelligence massage apparatus 100 may determine that the current massage operation being performed is strong for the user 1310. In addition, the artificial intelligence massage apparatus 100 may lower the massage intensity of the current massage operation and output a response speech such as “Lowering the massage intensity.” 1321. Alternatively, even if the user 1310 is not directly uttering the speech inputs to lower the massage intensity of the current massage operation via the user input unit 123 such as a remote controller, the artificial intelligence massage apparatus 100 may lower the massage intensity of the current massage operation and, if necessary, output a response speech such as “Lowering the massage intensity.” 1321.

The artificial intelligence massage apparatus 100 can generate training data for use in the learning of the massage operation determination model based on the current massage operation information, the user's facial expression information (or image data including the face of the user), and the massage operation control information of the user 1310. In addition, the massage operation determination model may be re-learned or updated using the generated training data. The artificial intelligence massage apparatus 100 may regard the massage operation controlled by the user 1310 being uttering as a speech or the massage operation controlled via the user input unit 123 by the user 1110 as an object massage operation.

Therefore, if the artificial intelligence massage apparatus 100 performs the same massage operation as the massage operation in the example of FIG. 13 later, and the user 1310 makes a painful facial expression 1311 or a frowning facial expression, even though there is no utterance or control input of the user 1310, the artificial intelligence massage apparatus 100 may output a query speech, such as “would you like to lower the massage intensity?” 1421 to lower the massage intensity of the current massage operation. If the user 1310 utters and agrees, the artificial intelligence massage apparatus 1310 may lower the massage intensity of the current massage operation.

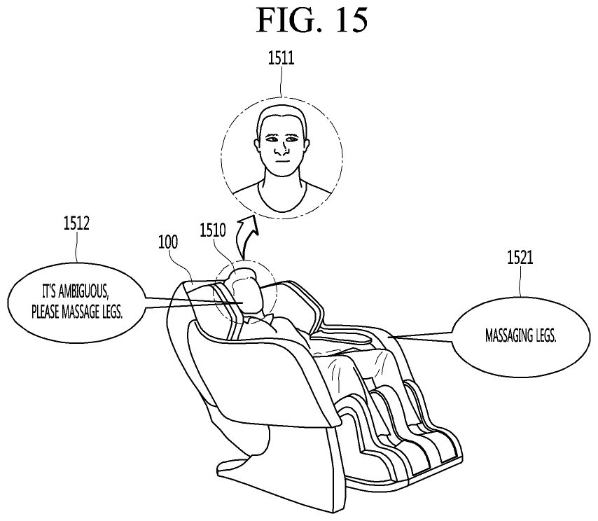

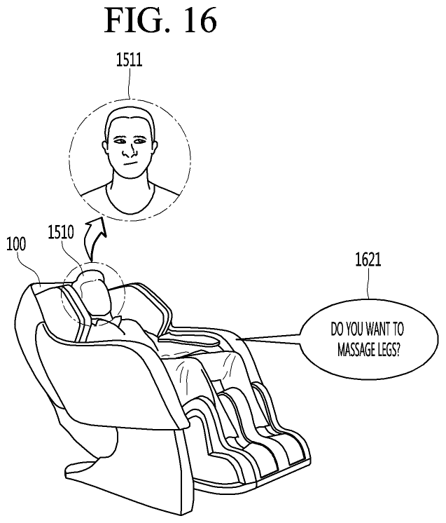

FIGS. 15 and 16 are views illustrating examples of controlling a massage operation in consideration of the facial expression or utterance of the user according to an embodiment of the present disclosure.

Referring to FIGS. 15 and 16, the user 1510 may receive a massage according to a preset massage operation or a massage operation set by the user 1510 using the artificial intelligence massage apparatus 100.

If the user 1510 utters a speech such as “it's ambiguous, please massage legs” 1512 while the user 1510 is dissatisfied with the current massage operation (for example, back massage) and makes an unpleasant facial expression 1511 or an unsatisfactory facial expression, the artificial intelligence massage apparatus 100 may determine that a massage operation currently being performed is unsatisfactory for the user 1510 based on the utterance speech 1512 of the user 1510. In addition, the artificial intelligence massage apparatus 100 may change the current massage operation to a leg massage operation and output a response speech such as “Massaging legs.” 1521. Alternatively, even if the user 1510 is not directly uttering as a speech but the user inputs to change the current massage operation into the leg massage operation via the user input unit 123 such as a remote controller, the artificial intelligence massage apparatus 100 may change the current massage operation to a leg massage, and if necessary, output a response speech such as “Massaging legs.” 1521.

The artificial intelligence massage apparatus 100 can generate training data for use in the learning of a massage operation determination model based on current massage operation information, user facial expression information (or image data including the face of the user), and the massage operation control information of the user 1510. In addition, the massage operation determination model may be re-learned or updated using the generated training data. The artificial intelligence massage apparatus 100 may regard the massage operation controlled by the user 1510 being uttering as a speech or the massage operation controlled via the user input unit 123 by the user 1110 as an object massage operation.

Therefore, if the artificial intelligence massage apparatus 100 performs the same massage operation as the massage operation in the example of FIG. 15 later, and the user 1510 makes an unpleasant facial expression 1511 or an unsatisfactory facial expression, even though there is no utterance or control input of the user 1510, the artificial intelligence massage apparatus 100 may output a query speech, such as “Do you want to massage legs?” 1621 in order to change the current massage operation to a leg massage operation. If the user 1510 utters and agrees, the artificial intelligence massage apparatus 1510 may change the current massage operation to a leg massage operation.

Examples of FIGS. 11 to 16 described above are not limited to the method for controlling the massage operation based on the facial expression of the user at a single time point. In other words, the massage operation model may determine the object massage operation based on image data including the face of the user for a predetermined period or the user's facial expression information for a predetermined period. In other words, the artificial intelligence massage apparatus 100 may determine the massage operation in consideration of not only the facial expression of the user at a single time point of the user, but also the change of the facial expression of the user for a predetermined period of time.

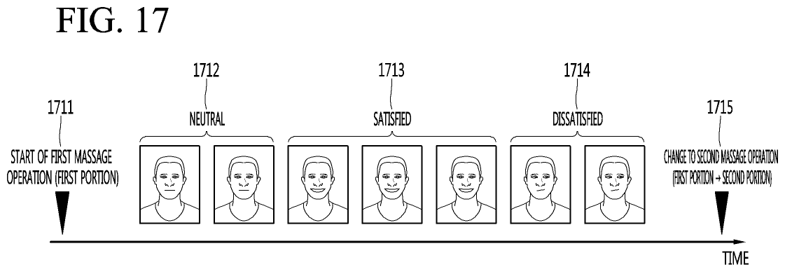

FIG. 17 is a view illustrating an example of controlling a massage operation in consideration of a change in the facial expression of a user according to an embodiment of the present disclosure.

Referring to FIG. 17, it is assumed that, after the artificial intelligence massage apparatus 100 starts (1711) the first massage operation on the first part, the facial expression of the user is changed in the order of the neutral facial expression 1712, the satisfactory facial expression 1713, and the unsatisfactory facial expression 1714, and the user changes the massage part from the first part to the second part through speech utterance or an input to the user input unit 123, thereby the massage operation has been changed 1715 from the first massage operation into the second massage operation. In this case, the massage operation determination model may be learned to change the first massage operation for the first massage part to the second massage operation for the second massage part when the first massage operation on the first massage part is being performed and the facial expression of the user is changed in the order of the neutral facial expression 1712, the satisfactory facial expression 1713, and the unsatisfactory facial expression 1714.

Later, if the user is being massaged with a first massage operation on the first massage part of the artificial intelligence massage apparatus 100 and the facial expression of the user is changing in order of neutral facial expression 1712, satisfactory facial expression 1713, and unsatisfactory facial expression 1714, the artificial intelligence massage apparatus 100 can ask whether to change the first massage operation for the first massage part to the second massage operation for the second massage part even though there is no separate input of the user.

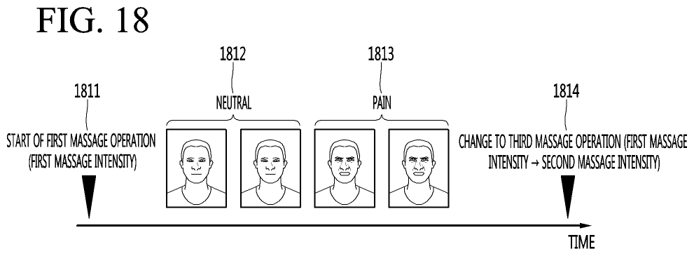

FIG. 18 is a view illustrating an example of controlling a massage operation in consideration of a change in the facial expression of a user according to an embodiment of the present disclosure.

Referring to FIG. 18, it is assumed that, after the artificial intelligence massage apparatus 100 starts 1811 a first massage operation having a first massage intensity, the facial expression of the user is changed in the order of a neutral facial expression 1812 and a painful facial expression 1813, and the user changed the first massage operation to the third massage operation 1814 by lowering the first massage intensity to the second massage intensity through speech utterance or input to the user input unit 123. In this case, the massage operation determination model may be learned to change the first massage operation of the first massage intensity to a third massage option of the second massage intensity when the first massage operation is being performed and the facial expression of the user is changed in the order of the neutral facial expression 1812 and the painful facial expression 1813.

Later, if the user is being massaged by the first massage operation of the first massage intensity of the artificial intelligence massage apparatus 100 and the facial expression of the user changes in the order of the neutral expression 1812 and the painful expression 1813, the artificial intelligence massage apparatus 100 may ask whether to change the first massage operation of the first massage intensity to the third massage operation of the second massage intensity even though there is no separate input of the user.

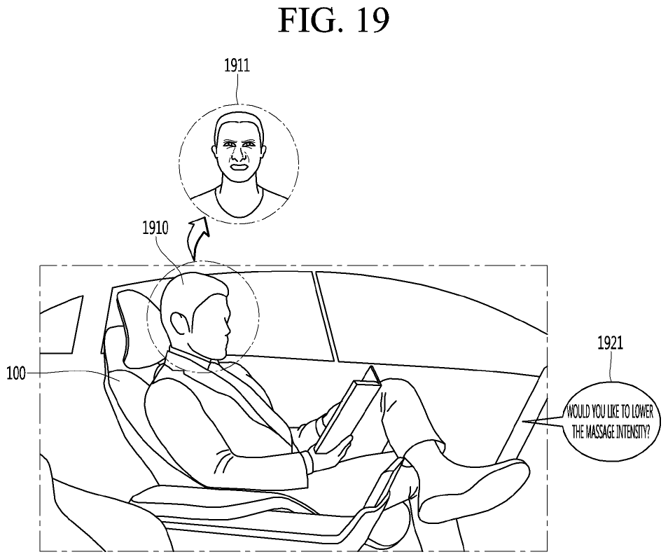

FIG. 19 is a view illustrating an example of controlling a massage operation according to an embodiment of the present disclosure.

Referring to FIG. 19, the artificial intelligence massage apparatus 100 may be a car seat and can be mounted on a self-driving vehicle or can be configured as a portion of the self-driving vehicle. Alternatively, the artificial intelligence massage apparatus 100 may be a self-driving vehicle, and the massage unit 160 may configure a car seat. In this case, the processor 180 of the artificial intelligence massage apparatus 100 may be a processor of a self-driving vehicle or may be a separate processor which is distinguished from the processor of the self-driving vehicle. If the processor 180 of the artificial intelligence massage apparatus 100 is distinguished from the processor of the self-driving vehicle, the processor 180 may operate in conjunction with the processor of the self-driving vehicle. The artificial intelligence massage apparatus 100 may be limited in operation to perform the massage operation only in a situation in which the self-driving vehicle is performing self-driving for safety.

The user 1910 may receive a massage according to a preset massage operation or a massage operation set by the user 1910 by using the artificial intelligence massage apparatus 100. If the user 1910 makes a painful facial expression 1911 or a frowning expression, the artificial intelligence massage apparatus 100 may output a query speech such as “Would you like to lower the massage intensity?” 1921 to lower the massage intensity of the current massage operation even though there is no utterance or control unit of the user 1910. If the user 1910 utters and agrees, the artificial intelligence massage apparatus 1910 may lower the massage intensity of the current massage operation.

According to various embodiments of the present disclosure, since the face of the user is recognized to determine whether the user is uttering, the massage operation can be controlled by speech even without wakeup word of the user.

In addition, according to various embodiments of the present disclosure, even though there is no user interaction, the massage operation may be controlled to be changed to a massage operation preferred by the user based on the current massage operation and the change of the facial expression of the user.

According to an embodiment of the present disclosure, the above-described method may be implemented as a processor-readable code in a medium where a program is recorded. Examples of a processor-readable medium may include hard disk drive (HDD), solid state drive (SSD), silicon disk drive (SDD), read-only memory (ROM), random access memory (RAM), CD-ROM, a magnetic tape, a floppy disk, and an optical data storage device.

What is claimed is:

1. An artificial intelligence massage apparatus for controlling a massage operation, comprising:
a microphone;
a camera;
a driver including at least one motor; and
a processor configured to:
receive image data including a face of a user via the camera,
in response to determining that the user is uttering a sound based on the image data, obtain speech data via the microphone,
generate intention information corresponding to the speech data, the intention information being associated with an intention of the user for controlling a massage of the user, and control a massage operation of the driver based on the intention information.

2. The artificial intelligence massage apparatus of claim 1, wherein the processor is further configured to:
in response to determining that the user is not uttering a sound based on the image data, determine an object massage operation based on the image data and current massage operation information, and
control the massage operation of the driver based on the object massage operation determined based on the image data and the current massage operation information.

3. The artificial intelligence massage apparatus of claim 2, wherein the processor is further configured to:
generate facial expression information from the image data based on a facial expression information generation model, and
determine the object massage operation based on the facial expression information and the current massage operation information based on a first massage operation determination model, and
wherein each of the facial expression information generation model and the first massage operation determination model includes an artificial neural network trained based on a machine learning algorithm or a deep learning algorithm.

4. The artificial intelligence massage apparatus of claim 3, wherein the first massage operation determination model determines the object massage operation based on facial expression information generated from image data of a predetermined section and massage operation information of the predetermined section.

5. The artificial intelligence massage apparatus of claim 3, wherein the processor is further configured to:
in response to the massage operation being controlled based on the intention information, generate first training data including the facial expression information, the current massage operation information, and control information of the massage operation corresponding to the intention information, and
wherein the first training data is used for training and updating the first massage operation determination model.

6. The artificial intelligence massage apparatus of claim 2, wherein the processor is further configured to:
determine the object massage operation from the image data based on a second massage operation determination model, and
wherein the second massage operation determination model includes an artificial neural network trained based on a machine learning algorithm or a deep learning algorithm.

7. The artificial intelligence massage apparatus of claim 6, wherein the second massage operation determination model determines the object massage operation based on image data of a predetermined section and massage operation information of the predetermined section.

8. The artificial intelligence massage apparatus of claim 6, wherein the processor is further configured to:
in response to the massage operation being controlled based on the intention information, generate second training data including the image data, the current massage operation information, and control information of the massage operation corresponding to the intention information, and
wherein the second training data is used for training and updating the second massage operation determination model.

9. The artificial intelligence massage apparatus of claim 1, wherein the processor is further configured to:
in response to the intention information not being associated with the control of the massage operation, determine an object massage operation based on the image data and current massage operation information, and
control the massage operation of the driver based on the object massage operation determined based on the image data and the current massage operation information.

10. The artificial intelligence massage apparatus of claim 1, wherein the artificial intelligence massage apparatus is mounted on a self-driving vehicle or configured as a portion of the self-driving vehicle.

11. The artificial intelligence massage apparatus of claim 10, wherein the processor is further configured to:
perform a massage operation while the self-driving vehicle is autonomously driving.

12. A method for controlling massage operation, the method comprising:
receiving image data including a face of a user via a camera;
in response to determining that the user is uttering a sound based on the image data, obtaining speech data via a microphone;
generating intention information corresponding to the speech data, the intention information being associated with an intention of the user for controlling a massage of the user; and
controlling a massage operation of a driver based on the intention information, the driver including at least one motor.

13. The method of claim 12, further comprising:
in response to determining that the user is not uttering a sound based on the image data, determining an object massage operation based on the image data and current massage operation information; and
controlling the massage operation of the driver based on the object massage operation determined based on the image data and the current massage operation information.

14. The method of claim 13, further comprising:
generating facial expression information from the image data based on a facial expression information generation model; and
determining the object massage operation based on the facial expression information and the current massage operation information based on a first massage operation determination model,
wherein each of the facial expression information generation model and the first massage operation determination model includes an artificial neural network trained based on a machine learning algorithm or a deep learning algorithm.

15. The method of claim 14, wherein the facial expression information is generated based on image data of a predetermined section, massage operation information of the predetermined section and the first massage operation determination model.

16. The method of claim 14, further comprising:
in response to the massage operation being controlled based on the intention information, generating first training data including the facial expression information, the current massage operation information, and control information of the massage operation corresponding to the intention information; and
training and updating the first massage operation determination model based on the first training data.

17. The method of claim 13, further comprising:
determining the object massage operation from the image data based on a second massage operation determination model,
wherein the second massage operation determination model includes an artificial neural network trained based on a machine learning algorithm or a deep learning algorithm.

18. The method of claim 12, wherein the driver is included in an artificial intelligence massage apparatus mounted on a self-driving vehicle or configured as a portion of the self-driving vehicle.

19. The method of claim 18, further comprising:
performing a massage operation via the driver while the self-driving vehicle is autonomously driving.

20. A non-transitory recording medium stored thereon a computer program for controlling a processor to perform a method of controlling a massage operation of a driver including at least one motor, the method comprising:
receiving image data including a face of a user via a camera;
in response to determining that the user is uttering a sound based on the image data, obtaining speech data via a microphone;
generating intention information corresponding to the speech data, the intention information being associated with an intention of the user for controlling a massage of the user; and
controlling a massage operation of the driver based on the intention information.
